(19) United States
(12) Patent Application Publication
(10) Pub. No.: US 2024/0411511 A1
(43) Pub. Date: Dec. 12, 2024

(54) VOICE-ACTIVATED CONTROL FOR DIGITAL OPTICAL SYSTEM

(71) Applicant: Alcon Inc., Fribourg (CH)

(72) Inventor: Douglas Allen, Santa Barbara, CA (US)

(73) Assignee: Alcon Inc., Fribourg (CH)

(21) Appl. No.: 18/667,003

(22) Filed: May 17, 2024

Related U.S. Application Data
(60) Provisional application No. 63/506,867, filed on Jun. 8, 2023.

(51) Int. Cl. G06F 3/16; A61B 90/00; A61B 90/20

(52) U.S. Cl. CPC G06F 3/167; A61B 90/20; A61B 90/37

(57) ABSTRACT

An electronic control unit (ECU) for a digital optical system includes a processor, a non-transitory computer-readable storage medium, and a translation engine. The performance settings include a focus-of-interest setting. Execution of the instructions by the processor causes the processor to receive voice commands from a user via a microphone during an ophthalmic procedure performed using the digital optical system. The voice commands are processed through the translation engine to convert the voice commands into a machine-readable instruction set. The processor adjusts the performance settings using the instruction set to change a state of the digital optical system, including transmitting display control signals to the display screen to overlay a reference grid onto a displayed digital image of a patient's eye. The focus-of-interest setting corresponds to a user-selected grid area of the reference grid.

### CROSS-REFERENCE TO RELATED APPLICATIONS

The present application claims the benefit of priority to U.S. Provisional Application No. 63/506,867 filed Jun. 8, 2023, which is hereby incorporated by reference in its entirety.

### INTRODUCTION

The present disclosure relates to hardware and related strategies or methodologies for controlling a digital optical system, in particular one having a microscope.

As appreciated in the art, a digital optical system enables a surgeon to view a patient's ocular anatomy under high levels of magnification. Magnified viewing of the patient's eye is typically provided by a microscope appended to an articulated serial robot arm. The microscope for its part includes an optical head containing optical lenses and a controllable light source. Ophthalmic microscopes also include one or more eyepieces or oculars through which the surgeon can view the magnified images. The magnified images are also displayed on a high-resolution display screen, for instance when a digital camera is attached to an analog microscope. Adjustment of the various modes and control settings of the microscope and is peripheral devices is typically performed via a set of user interface devices, e.g., foot-operated pedals, hand-adjusted knobs or buttons, touch screens, and other user-manipulated devices.

### SUMMARY

Disclosed herein are automated systems and methods for providing voice control functionality of a digital optical system, e.g., within an ophthalmic surgical suite. The present solutions address limitations commonly associated with the aforementioned foot-operated or hand-operated user input devices. In lieu of such analog devices, the present teachings rely on simple voice commands to control settings of the digital optical system in a “hands-free” manner. Touch-free voice control also minimizes or eliminates the need for physical interaction between the user and the digital optical system when the user is required to adjust the performance settings.

As appreciated in the art, certain surgical procedures such as cataract surgery—absent complications or problematic patient anatomy or conditions—can require several minutes to complete. In contrast, analog adjustment of the performance settings of a digital optical system during such procedures could take ten seconds or more to complete, which is a significant portion of the total surgery time. Given the relative brevity of the surgical procedure, a delay of this magnitude can be unacceptable from the perspective of both the surgeon and the patient. In contrast, the voice command-based control strategy set forth herein could reduce the settings adjustment time, e.g., to several milliseconds. As an added benefit, the surgeon's hands remain free to perform surgical maneuvers while eliminating physical handling of the digital optical system and possible cross-contamination of its associated surfaces.

Accordingly, an electronic control unit (ECU) is disclosed herein for use with a digital optical system having a display screen of the type summarized above. An embodiment of the ECU includes a processor and a non-transitory computer-readable storage medium/memory on which instructions are recorded for controlling performance settings of the digital optical system. The performance settings as contemplated herein include at least a focus-of-interest setting. The performance settings could also include, e.g., digital zoom, depth-of-field, lighting, and/or other application-specific performance settings.

Execution of the instructions by the processor causes the processor to receive voice commands from a surgeon, medical support staff, or another user in the ophthalmic surgical suite. This action occurs with the assistance of a microphone during an ophthalmic procedure performed using the digital optical system. Execution of the instructions also causes the processor to process the received voice commands through a translation engine to convert the voice commands into machine-readable instructions, typically alphanumeric characters or text.

The processor thereafter executes the machine-readable instructions to adjust the performance settings of the digital optical system and thereby change a present state of the digital optical system. This action may include transmitting electronic display control signals to the display screen to cause the display screen to overlay a reference grid onto a displayed digital image of the patient's eye. The focus-of-interest setting in this case would correspond to a user-selected grid area of the reference grid.

The voice commands in one or more embodiments could include a predetermined primary focus utterance of the user. In this case, the processor could overlay the reference grid onto the displayed image of the patient's eye in response to the primary focus utterance. The voice commands could also include a predetermined secondary focus utterance of the user. In response to the predetermined secondary utterance, the processor may set the grid area and stop presenting the reference grid.

The reference grid in one or more embodiments could be constructed as a rectilinear grid having rectangular grid cells. The rectangular grid cells in a possible implementation may be arranged in at least five rows and at least five columns, typically but not necessary with an equal number of rows and columns. Alternatively, the reference grid could be a curvilinear grid having non-rectangular grid cells.

The performance settings as contemplated herein may optionally include a digital zoom setting. The processor in such a configuration could automatically adjust the digital zoom setting in response to a predetermined zoom utterance of the user. In another possible implementation, the performance settings could include a depth-of-field function, with the processor in this case being configured to command a depth-of-field setting of the digital optical system in response to a predetermined depth-of-field utterance of the user.

The digital optical system may also include a light source, in which case the performance settings could include a desired light setting of the light source. The processor in such an embodiment would command a desired light setting of the light source in response to a predetermined light setting utterance of the user. For instance, the desired light setting could include a brightness level of the light source. The light source in some embodiments may include multiple coaxial light sources and an oblique light source. The desired light setting in such a construction may include a selection of the coaxial light sources or the oblique light source, or other light setting such as a particular color or wavelength of emitted light.

The processor could also be programmed with one or more default settings of the digital optical system for each respective one of the performance settings, and to automatically select the default settings in response to a predetermined default utterance of the user. The predetermined default utterance may include, e.g., a name of the user and/or a name of a hospital or medical facility in which the digital optical system is employed, for instance a teaching hospital.

Also disclosed herein is a visualization system that includes the digital optical system, a microphone, and the above-summarized ECU.

Another aspect of the disclosure includes a method for controlling performance settings of the digital optical system. An embodiment of the method includes receiving voice commands from a user during an ophthalmic procedure via the microphone, processing the voice commands through a translation engine of the ECU to thereby convert the voice commands into a machine-readable instruction set, and then adjusting the performance settings of the digital optical system via a processor of the ECU. This action occurs using the machine-readable instruction set, which in turn changes a state of the digital optical system.

Adjusting the performance settings of the digital optical system includes transmitting display control signals to the display screen to cause the display screen to overlay a reference grid onto a displayed digital image of a patient's eye. The focus-of-interest setting corresponds to a user-selected grid area of the reference grid as summarized above.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 is an illustration of a representative ophthalmic surgical suite having a digital optical system in which a microscope is equipped with the voice control capabilities described herein.

FIG. 2 is a schematic illustration of an electronic control system for use with the digital optical system shown in FIG. 1.

FIGS. 3A and 3B illustrate alternative configurations of a reference grid usable as part of the present methodology.

FIG. 4 is a flow chart describing an exemplary method for controlling the digital optical system of FIG. 1 using the electronic control system illustrated in FIG. 2.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

Embodiments of the present disclosure are described herein. It is to be understood, however, that the disclosed embodiments are merely examples and other embodiments can take various and alternative forms. The figures are not necessarily drawn to scale. Some features could be exaggerated or minimized to show details of particular components. Therefore, specific structural and functional details disclosed herein are not to be interpreted as limiting, but merely as a representative basis for teaching one skilled in the art to variously employ the present disclosure.

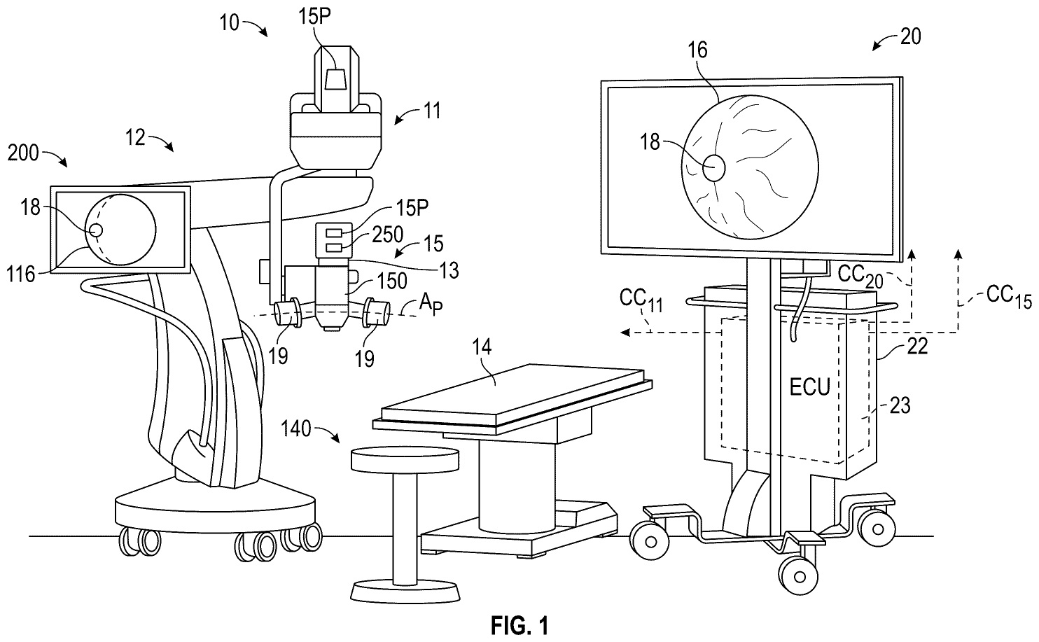

Referring to the drawings, wherein like reference numbers refer to like components, a representative ophthalmic surgical suite 10 is illustrated in FIG. 1. The ophthalmic surgical suite 10 is equipped with a digital optical system 11 having an electronic control unit (ECU) 23 as described in detail below. The digital optical system 11 may be connected to/supported by a serial robot arm 12 and positioned proximate an operating table 14. During a vitreoretinal, cataract, or other surgical procedure within the suite 10, a patient (not shown) may be positioned on the operating table 14 or on another suitable platform, with a surgeon (not shown) seated on a stool 140. Although omitted from FIG. 1 for the purpose of illustrative simplicity, respective heights of the operating table 14 and the stool 140 could be adjusted with the assistance of knobs, levers, or foot pedals in a typical implementation as noted above.

Within the ophthalmic surgical suite 10 of FIG. 1, visualization of a patient's eye (not shown) performed before or concurrent with an ophthalmic procedure entails use of a microscope 15 of the digital optical system 11. An articulated serial robot arm 12 could be securely connected to the microscope 15 to maneuver and support the microscope 15. Such a microscope 15 allows the surgeon to view a patient's ocular anatomy under high magnification. For instance, using associated hardware and software, the surgeon is able to view highly magnified images 16 and 116, e.g., of a retina 18. Viewing in one or more embodiments is facilitated by a high-resolution display screen 20 upon proper positioning an optical head 150 of the microscope 15. A smaller additional display screen 200 could be positioned elsewhere in the suite 10 to facilitate viewing by other medical personnel during the procedure. Information presented via the displays 20 and 200 is controllable by electronic display control signals CC₂₀ from the 23 as described below.

As appreciated by those skilled in the art, an ophthalmic microscope such as the microscope 15 illustrated in FIG. 1 consists of several main components. For instance, the optical head 150 contains the various lenses and optics suitable for magnifying a patient's eye. The microscope 15 also includes handles 19 and a controllable light source 250 having controllable light settings as described below. Such a light source 250 may be equipped with coaxial light sources 250A and/or an oblique light source 250B in some embodiments. The optical head 150 could also contain an objective lens (not shown) providing different magnifications, and one or more upwardly-angled eyepieces or oculars (not shown) through which the surgeon can view the magnified images 16, 116.

As contemplated herein, there are three possible use scenarios of for the microscope 15: (i) fully analog, during which a surgeon looks through the oculars, (ii) hybrid, during which a digital camera 13 is mounted to one of the sets of oculars of an analog embodiment of the microscope 15, and during which the surgeon can look at a monitor to see the digital image or look through the oculars, and (iii) fully digital, in which the surgeon can only view the digital image. While the present approach is not limited to any particular use scenario, the solutions provided herein are particularly suited to use with the hybrid version, although it should apply to the fully digital version as well.

Also present within the exemplary ophthalmic surgical suite 10 of FIG. 1 is a cabinet 22 containing the ECU 23. It is also envisioned that the ECU 23 could be located in a base or other suitable portion of the serial arm 12. Likewise, the display screen 20 could be alternatively embodied as three-dimensional (3D) glasses, one or more wall-mounted monitors, and/or another application-suitable visualization device or devices. The ECU 23 may be equipped in hardware and programmed in software, i.e., configured, to coordinate electronic features and settings of the optical head 150 and/or other equipment or payloads used within the suite 10. Control of the performance settings of the microscope 15 via the ECU 23 occurs by transmitting electronic control signals (CC₁₅) to a resident control processor 15P of the microscope 15, with the control processor 15P in turn controlling various performance settings of the microscope 15 as set forth below. The cabinet 22 for its part is optional, and could be constructed of a lightweight and easily sanitized construction, e.g., painted aluminum or stainless steel, and used to protect constituent hardware of the ECU 23 from possible ingress of dust, debris, and moisture.

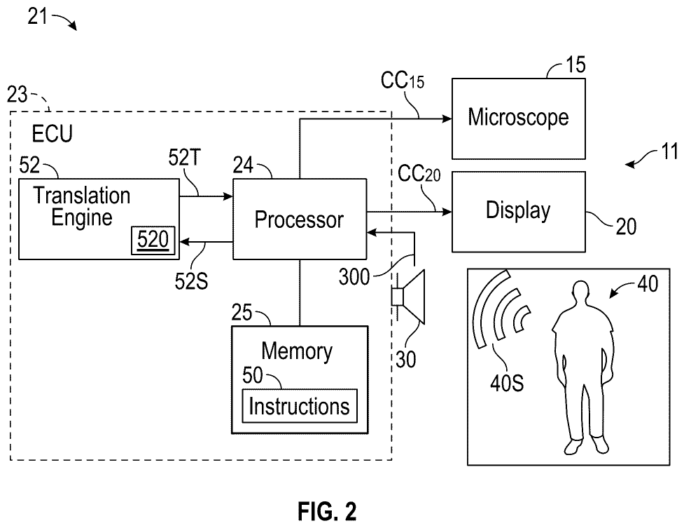

Referring now to FIG. 2, an ophthalmic system 21 is shown schematically to include the ECU 23 and the digital optical system 11 of FIG. 1, including at least the microscope 15 and microphone 30. In general, the ECU 23 includes one or more processors 24 and a computer-readable storage medium/memory 25 containing computer-readable/executable instructions 50 for controlling the performance settings of the digital optical system 11 shown in FIG. 1, including at least a focus-of-interest setting as set forth in detail below. The ECU 23 also includes a translation engine 52 in communication with the processor(s) 24. As appreciated in the art, such a translation engine 52 is configured to process received spoken voice commands 40S from a user 40, e.g., the above-noted surgeon or attending medical staff, in such a manner that ECU 23 is able to accurately discern the intent of the user 40.

The processor 24 and the translation engine 52 together utilize speech recognition software 520 to ultimately convert the voice commands 40S (“utterances”) into alphanumeric machine-executable instructions 52T, which the translation engine 52 then interprets as instructions from the user 40. In general, the voice commands 40S are initially detected using a suitably configured microphone 30, e.g., a condenser, dynamic, or ribbon microphone, or simple USB microphone. The microphone 30, which may be connected to/integrated with the microscope 15 or positioned at a suitable location within the ophthalmic surgical suite 10 of FIG. 1, outputs audio detection signals 300 to the processor 24. The processor 24 then digitally processes the utterances as needed, e.g., using digital signal processing techniques and filters to remove background noise in the audio detection signals 300, normalize its audio levels, etc. The processor 24 thereafter outputs a filtered audio signal 50S to the translation engine 52.

The translation engine 52 for its part receives the filtered audio signal 52S and thereafter performs speech recognition functions using the speech recognition software 520, e.g., feature extraction and/or language modeling, possibly with the assistance of artificial intelligence tools such as neural networks in order to increase speech recognition accuracy. As part of the present approach, the translation engine 52 could output corresponding alphanumeric data as the machine-readable instruction set 52T. The memory 25 of FIG. 2 may be programmed with associated control logic for the microscope 15. Utterances of the user 40 of FIG. 2 are thereafter used by the processor 24, seamlessly and with minimal latency, to control the operation of the digital optical system 11 illustrated in FIG. 1.

Execution of the instructions 50 by the processor 24 of FIG. 2 thus causes the processor 24 to receive the voice commands 40S from the user 40 via the microphone 30 during an ophthalmic procedure. As part of this process, the processor 24 feeds or processes the voice commands 40S through the translation engine 52 and its speech recognition software 520 as described above to thereby convert the voice commands 40S into the machine-readable instruction set 52T. Non-limiting exemplary utterances are described below to provide just some of the many possible voice commands 40S that could be used with the digital optical system 11 of FIG. 1. Ultimately, the processor 24 adjusts the performance settings of the digital optical system 11 in real-time using the machine-readable instruction set 52T. In this manner, the processor 24 changes a logic state, operating mode, or other state of the digital optical system 11. The processor 24 may also transmit the electronic microscope control signals CC₁₅ to the microscope 15 and/or the electronic display control signals CC₂₀ to the display screen 20 of FIG. 1.

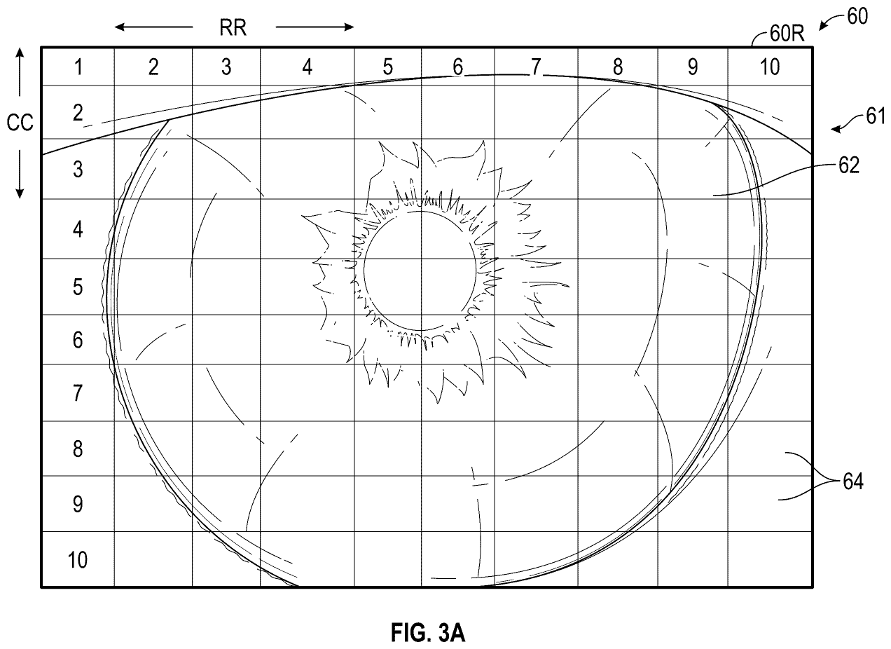

Referring now to FIG. 3A, transmission of the electronic display control signal CC₂₀ to the display screen 20 illustrated in FIG. 1 would cause the display screen 20 to overlay a reference grid 60 onto a displayed digital image 61 of the patient's eye 62. The above-noted focus-of-interest setting as contemplated herein corresponds to a user-selected grid area of the reference grid 60. The particular shape of the reference grid 60 could vary with the application. For instance, the reference grid 60 is depicted as a rectilinear grid 60R in FIG. 3A, i.e., arranged in multiple rows RR and multiple columns CC, such that the rectilinear grid 60R has a plurality of rectangular grid cells 64. In a representative embodiment, the rectangular grid cells 64 could be arranged in at least five rows RR and at least five columns CC, e.g., a 10×10 grid as shown. Other embodiments could include a different number of rows and or columns, and therefore the 10×10 grid is just one possible implementation.

Alternatively as illustrated in FIG. 3C, an alternative curvilinear reference grid 60C could be constructed as a curvilinear grid 60C, with such a reference grid having non-rectangular grid cells. In such an implementation, the curvilinear grid 60C could be divided into multiple radial sections 65, which are labeled 1R, 2R, . . . , 12R for clarity in the illustrated non-limiting twelve-section embodiment. In turn, each of the radial sections 65 may be divided into a plurality of non-rectangular grid cells 640. The non-rectangular grid cells 640 are labeled 1-7 in FIG. 3B for a representative seven-cell embodiment of the radial sections 65. More or fewer radial sections 65 and/or non-rectangular grid cells 640 may be used in other implementations, and therefore the exemplary embodiments of FIGS. 3A and 3B are non-limiting.

Voice Commands: referring once again to FIGS. 1 and 2, exemplary voice commands or utterances of the user 40 may be used to control a wide range of functions. For instance, the user 40 may control internal camera operations of the microscope 15 such as zoom, focal area-of-interest, depth-of field, light levels, light temperature, etc. Similarly, the user 40 could use voice commands to control left-right/xy motion of the optical head 150 as perceived via the display screen 20 and/or 200, up-down/y-axis motion of the optical head 150, in/out focus, and other possible functions.

Focus: typically, an autofocus routine for the microscope 15 would be aimed at a center of the display screen 20. However, this may not be the most desirable area to bring into focus. Voice control of focus can therefore be responsive to a predetermined primary focus utterance. For example, the user 40 could speak an intuitively descriptive word or phrase such as “Focus”. The processor 24 in such an example use case may be configured to overlay the reference grid 60 or 60C of FIGS. 3A and 3B onto the displayed image of the patient's eye 62 (see FIG. 3A) in response to such a predetermined primary focus utterance.

The voice commands could also include a predetermined secondary focus utterance of the user 40, with the secondary focus utterance providing more detail or specificity of the action commanded via the primary focus utterance. For example, in response to the predetermined secondary utterance, the processor 24 could set the user-selected grid area of FIG. 2 and stop presenting the reference grid 60 or 60C of FIGS. 3A and 3B. An example of secondary of a secondary focus utterance within the scope of the disclosure could be spoken numerals “4 3”, with “4” and “3” in this illustrative example corresponding to a particular grid cell 64 or 640 in the reference grid 60 or 60° C. of respective FIGS. 3A and 3B. The user 40 could momentarily view an anatomical feature that may not be fully in focus by bringing the feature into focus via voice commands, then uttering a phrase such as “Focus Return” to return to the previous focal area-of-interest.

Zoom: the performance settings adjusted using the voice commands 40S of FIG. 2 could include a digital zoom setting of the digital optical system 11 shown in FIG. 1. In this case, the processor 24 would adjust the digital zoom setting in response to a predetermined zoom utterance of the user 40. For instance, the user 40 could utter the phrase “Zoom 10” to command a 10× digital zoom level, with possible zoom levels of 1, 2, . . . , N also being possible options, up to functional limits of the digital optical system 11. Here, N may be an integer zoom, e.g., 3 on a nominal 0-5 scale where 0=0% zoom, 3=60% zoom, and 5=100% zoom, or a fractional zoom number such as 3.5 to provide increased zoom control precision. It is also possible to utter phrases such as “Continuous Zoom N” to zoom in a smooth fashion from the current level to the commanded level, i.e., N.

Adjustment speed is set herein by user preference rather than being driven by any physical capabilities of a moving lens system, which is typically designed for high resolution analog motion as opposed to zoom speed. In addition to increased response time, voice control of the various functions as contemplated herein has additional advantages. All have to do with the elimination of certain adjustment features from a foot switch mechanism. As appreciated in the art, a foot switch provides zoom control capabilities among several associated functions. Removing this function from the foot switch greatly reduces the required complexity of the foot switch, which in some cases could still be retained for other less detailed tasks than controlling the microscope 15.

Additionally, whenever a surgeon or other user 40 activates a typical foot switch, the foot motion tends to travel through the surgeon's body to the surgeon's hands, and thus to any instruments or tools the surgeon might be holding. Also, the myriad functions of a foot switch are mapped differently from surgeon to surgeon. In contrast, the present voice command-based method is universal, i.e., the voice control commands could be standardized across a wide range of possible users 40. This feature in turn simplifies the structure and workflow within the ophthalmic surgical suite 10 of FIG. 1.

Depth-of-Field: analog microscopes and certain other cameras typically use a mechanical slider to change an iris aperture and thereby change the depth-of-field, i.e., a distance range over which a target remains in focus. The slider is set at a certain percentage, for example 30% of full open, and thereafter maintained in this position. Changes to depth-of-field using such a setup requires a physical translation of the mechanical slider, which in turn requires an adjustment in light intensity to match the new iris opening. As this is not practically performed in an operating room environment, it is more common for a surgeon to balance depth-of-field and resolution preferences at the onset of a given procedure and thereafter leave the slider in a fixed position. Depth-of-field is inversely related to resolution.

There are times, such as during an internal limiting membrane (ILM) peel, where the surgeon may wish to reduce depth-of-field to thereby gain greater resolution. This tradeoff is accomplished herein using voice commands. As appreciated in the art, the ILM is an approximately 3-micron thick tissue that a surgeon is sometimes required to peel off of the retina. The edge of the ILM can be floating above the retina, and therefore is difficult to see during the ILM peel maneuver. Autofocus of a typical microscope will not readily converge on the edge of the ILM tissue. Instead, autofocus will find a more substantial structure as the point of focus. Given that this is the time where the lowest depth-of-field (i.e., highest resolution) would be used, the user might wish to command an area of interest focus via the grid described herein, but also the ability to move the focal point forward or back in a small increment to better bring the edge into focus.

Therefore, in accordance with an aspect of the disclosure the performance settings of the digital optical system 11 of FIG. 1 may include a depth-of-field function. The processor 24 of FIG. 2 in such an embodiment may command a depth-of-field setting of the digital optical system 11 in response to a predetermined depth-of-field utterance of the user 40. As with the above-described zoom function, the user 40 could speak a simple phrase such as “Depth 5” to command a nominal or relative depth-of-field of “5”,e.g., a middle-of-range setting, with other numeric levels such as 1, 2, etc., corresponding to digital increments and limits of the digital optical system 11. In embodiments in which the light source 250 has variable light temperature, each successive change in the depth-of-field, the processors could also change the light temperature.

Lighting: also as shown in FIG. 1 and noted above, and further with respect to the light source 250, voice-controlled performance settings could also include a desired light setting of the light source 250. The processor 24 in one or more embodiments may be configured to command a desired light setting of the light source 250 of FIG. 1 in response to a predetermined light setting utterance of the user 40. For example, the user 40 could command a brightness level of the light source 250, e.g., using a spoken phrase such as “Light 10”, or similarly “Light 1”, “Light 2”, etc., up to the limits of the lighting system. Alternatively, a phrase such as “Light Up” and “Light Down” could be spoken by the user 40 to cause the processor to increase/decrease the light level by one step.

As the light source 250 of FIG. 1 could optionally include the multiple coaxial light sources 250A and the oblique light source 250B, the desired light setting in one or more embodiments may include a selection of the coaxial light sources 250A or the oblique light source 250B, for instance by uttering the terms “Coaxial” or “Oblique” as desired. Likewise, the light source 250 could have different colors, wavelengths, color temperatures, or other characteristics such as pre-set contrast/filter/light settings that help highlight the anatomy of the patient's eye in a more vivid fashion than basic illumination. The user 40 could similarly adjust such settings using the voice commands 40S of FIG. 2. For instance, when near-infrared light is required, the user 40 may say “IR”, to which the light source 250 might respond by switching to infrared, e.g., from the visible light spectrum. Within the scope of the disclosure, the user 40 could also select pre-defined surgeon preference profiles, possibly including unique combinations of colors for each surgeon and/or surgical step.

In lieu of or as a concurrently programmed alternative selection possibility, the processor 24 could be programmed with one or more default settings of the digital optical system 11. This may be true for each respective one of the performance settings. The processor 24 could then automatically select the default settings in response to a predetermined default utterance of the user 40 depicted in FIG. 2. One possible implementation of this optional programmed feature could include a predetermined default utterance in which the user 40 speaks aloud the user's name or, if the user 40 is not the surgeon, the name of the particular surgeon performing the procedure. As another option, the user 40 could speak the name of a hospital or medical facility, e.g., a teaching hospital. Such an approach would set the digital optical system 11 to a hospital-specific standard, which in turn may help ensure consistency across a wide number of surgeons or students.

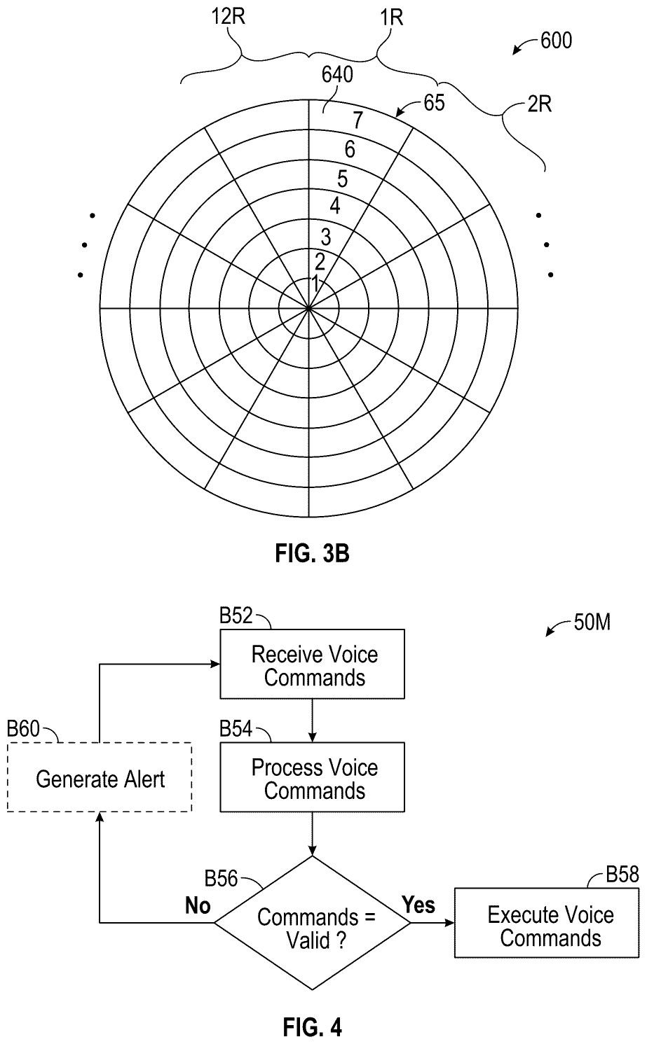

Referring now to FIG. 4, a method 50M in accordance with an embodiment is described for simplicity in terms of corresponding code segments or logic/terminal blocks. In practice, the instructions 50 which embody the method 50M and reside in the memory 25 of FIG. 2 are arranged into such blocks and executed by the processor 24. e.g., when used within the ophthalmic surgical suite 10 illustrated in FIG. 1.

Beginning with logic block B52 (“Receive Voice Commands”), the microphone 30 receives the voice commands 40S from the user 40 as illustrated in FIG. 2. The method 50M proceeds to block B54 as the microphone 30 outputs the audio detection signals 300 to the processor 24.

At block B54 (“Process Voice Commands”), the processor 24 responds to the audio detection signals 300 by processing the voice commands, as captured by the audio detection signals 300, through the translation engine 52 of FIG. 2. Block B54 includes converting the voice commands into the machine-readable text 52T, for instance by converting the audio detection signals 300 into corresponding alphanumeric words or representative character strings. The method 50M thereafter continues to block B56.

Block B56 (“Commands=Valid?”) includes comparing the machine-readable text 52T to a predetermined voice commands, such as a list of commands or acceptable variations thereof stored in an accessible library in memory 25 of FIG. 2. The method 50M proceeds to block B58 when the voice commands match one of the predetermined voice commands in the validated library. The method 50M proceeds in the alternative to block B60 when the voice commands do not match any of the predetermined voice commands.

At block B58 (“Execute Voice Commands”) the processor 24 performs the commanded action as detected in block B54. For example, if the user 40 of FIG. 2 utters the phrase “Zoom 5”, and the phrase “Zoom 10” has been stored in the above-noted library as a valid command, the processor 24 proceeds to perform the commands zoom action. For example, the processor 24 may adjust the zoom performance setting of the digital optical system 11 in this example scenario, using the machine-readable text 52T to change the state of the digital optical system 11. The change of state may entail transmitting the electronic display control signals CC20 to the display screen 20 and/or 200 to cause the display screen 20 and/or 200 to overlay the reference grid 60 or 60C of FIGS. 3A and 3B, respectively, onto the displayed digital image of the patient's eye 62 (FIG. 3A). The method 50M is thus complete, resuming anew with block B52.

Block B60 (“Generate Alert”) is performed when the processor 24 detects an utterance of the user 40 in block B56 that does not correspond to a predetermined or validated spoken word/phrase. In response, the processor 24 could generate a suitable alert indicating that the word/phrase is not recognized, such as but not limited to presenting a text alert on the display screen 20, sounding an auditory alert, activating a haptic alert, etc. Alternatively, the processor 24 could display a list of acceptable voice commands or broadcast a message requesting that the user 40 speak louder or try a different word/phrase. The method 50M then resumes with block B52.

The solutions as presented herein save time within the ophthalmic surgical suite 10 illustrated in FIG. 1 by using voice commands as opposed to using a foot switch or manual analog adjustments. Use of voice commands to control the microscope 15 as envisioned herein does not disturb the sterile field. Elimination of foot switch-based control of certain functional settings of the microscope 15 and the need for the user 40 of FIG. 2 to learn its related programmed functions reduces complexity and helps keeps the surgeon's hands steady.

Moreover, the digital optical system 11 of FIG. 1 could be commanded back into operation and default settings after storage or power-off states using predetermined voice commands, e.g., “Initialize” or “Ready State”. The provided functionality collectively saves time and effort between procedures being performed in the suite 10. As a result, the present solutions have the benefit of yielding increased nimbleness and speed of adjustment when controlling the digital optical system 11 illustrated in FIG. 1. These and other attendant benefits will be readily appreciated by those skilled in the art in view of the foregoing disclosure.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

What is claimed is:

1. An electronic control unit (ECU) for a digital optical system having a display screen, comprising:
a processor;
a non-transitory computer-readable storage medium on which instructions are recorded for controlling performance settings of the digital optical system, the performance settings including a focus-of-interest setting; and
a translation engine in communication with the processor, wherein execution of the instructions by the processor causes the processor to:
receive voice commands from a user via a microphone during an ophthalmic procedure performed using the digital optical system;
process the voice commands through the translation engine to thereby convert the voice commands into a machine-readable instruction set; and
adjust the performance settings of the digital optical system using the machine-readable instruction set to thereby change a state of the digital optical system, including transmitting electronic display control signals to the display screen to cause the display screen to overlay a reference grid onto a displayed digital image of a patient's eye, wherein the focus-of-interest setting corresponds to a user-selected grid area of the reference grid.

2. The ECU of claim 1, wherein the voice commands include a predetermined primary focus utterance of the user, and wherein the processor is configured to overlay the reference grid onto the digital image of the patient's eye in response to the predetermined primary focus utterance.

3. The ECU of claim 2, wherein the voice commands include a predetermined secondary focus utterance of the user, and wherein in response to the predetermined secondary utterance, the processor is configured to set the user-selected grid area and stop presenting the reference grid.

4. The ECU of claim 1, wherein the reference grid is a rectilinear grid having rectangular grid cells.

5. The ECU of claim 1, wherein the reference grid is a curvilinear grid having non-rectangular grid cells.

6. The ECU of claim 1, wherein the performance settings include a digital zoom setting of the digital optical system, and wherein the processor is configured to adjust the digital zoom setting in response to a predetermined zoom utterance of the user.

7. The ECU of claim 1, wherein the performance settings include a depth-of-field function, and wherein the processor is configured to command a depth-of-field setting of the digital optical system in response to a predetermined depth-of-field utterance of the user.

8. The ECU of claim 7, wherein the digital optical system includes a light source, and wherein the processor is configured to automatically adjust a color temperature of the light source with each successive change in the depth-of-field setting.

9. The ECU of claim 1, wherein the digital optical system includes a light source, the performance settings include a desired light setting of the light source, and the processor is configured to command a desired light setting of the light source in response to a predetermined light setting utterance of the user.

10. The ECU of claim 9, wherein the desired light setting includes a brightness level of the light source.

11. The ECU of claim 9, wherein the light source includes multiple coaxial light sources and an oblique light source, and wherein the desired light setting includes a selection of the coaxial light sources or the oblique light source.

12. The ECU of claim 1, wherein the processor is programmed with one or more default settings of the digital optical system for each respective one of the performance settings, and is to automatically select the default settings in response to a predetermined default utterance of the user.

13. The ECU of claim 12, wherein the user is a surgeon that will perform the ophthalmic procedure, and the predetermined default utterance includes a name of the surgeon.

14. The ECU of claim 12, wherein the predetermined default utterance includes a name of a hospital or medical facility.

15. A visualization system comprising:
a digital optical system having a digital microscope and a display screen;
a microphone; and
an electronic control unit (ECU) comprising:
a processor in communication with the digital optical system and the microphone;
a non-transitory computer-readable storage medium on which instructions are recorded for controlling performance settings of the digital optical system, the performance settings including a focus-of-interest setting; and
a translation engine in communication with the processor, wherein execution of the instructions by the processor causes the processor to:
receive voice commands from a user via the microphone during an ophthalmic procedure, wherein the ophthalmic procedure is performed using the digital optical system;
process the voice commands through the translation engine to thereby convert the voice commands into a machine-readable instruction set; and
adjust the performance settings of the digital optical system using the machine-readable instruction set to thereby change a state of the digital optical system, including transmitting display control signals to the display screen to cause the display screen to overlay a reference grid onto a displayed digital image of a patient's eye, wherein the focus-of-interest setting corresponds to a user-selected grid area of the reference grid.

16. The visualization system of claim 15, wherein the voice commands include a predetermined primary focus utterance of the user, and wherein the processor is configured to overlay the reference grid onto the displayed image of the patient's eye in response to the predetermined primary focus utterance.

17. The visualization system of claim 16, wherein the reference grid is a rectilinear grid having rectangular grid cells.

18. The visualization system of claim 16, wherein the performance settings include a digital zoom setting of the digital optical system, and wherein the processor is configured to adjust the digital zoom setting in response to a predetermined zoom utterance of the user.

19. A method for controlling performance settings of a digital optical system, comprising:
receiving voice commands from a user during an ophthalmic procedure via a microphone, wherein the ophthalmic procedure is performed using the digital optical system;
processing the voice commands through a translation engine of an electronic control unit (ECU) to thereby convert the voice commands into a machine-readable instruction set; and
adjusting one or more performance settings of the digital optical system via a processor of the ECU using the machine-readable instruction set to thereby change a state of the digital optical system, the performance settings including a focus-of-interest setting, wherein adjusting the performance settings of the digital optical system includes transmitting display control signals to the display screen to cause the display screen to overlay a reference grid onto a displayed digital image of a patient's eye, and wherein the focus-of-interest setting corresponds to a user-selected grid area of the reference grid.

20. The method of claim 19, wherein the voice commands include a predetermined primary focus utterance of the user and a secondary focus utterance of the user, and wherein the processor is configured to overlay the reference grid onto the displayed image of the patient's eye in response to the predetermined primary focus utterance.
